(19) United States
(12) Patent Application Publication
(10) Pub. No.: US 2025/0044616 A1
(43) Pub. Date: Feb. 6, 2025

(54) FOUR-AXIS ASYMMETRIC ORTHOKERATOLOGY LENS AND MANUFACTURING METHOD

(71) Applicant: Shanghai FULO Medical Instrument Co., Ltd., Shanghai (CN)

(72) Inventors: Baoguang JIANG, Shanghai (CN); Mototora KAI, Shanghai (CN); An LI, Shanghai (CN)

(73) Assignee: Shanghai FULO Medical Instrument Co., Ltd., Shanghai (CN)

(21) Appl. No.: 18/920,313

(22) Filed: Oct. 18, 2024

(30) Foreign Application Priority Data
Feb. 29, 2024 (CN) 202410228718.X
Sep. 6, 2024 (CN) 202411253643.7

(51) Int. Cl. G02C 7/04

(52) U.S. Cl. CPC G02C 7/047

(57) ABSTRACT

A four-axis asymmetric orthokeratology lens includes a base curve zone, and a reverse curve zone, an adaptation curve zone, and a peripheral curve zone that are successively formed outward from a periphery of the base curve zone. A center of a circle of the base curve zone corresponds to a center of a cornea, and a zone, corresponding to the adaptation curve zone, of the cornea is defined as a specific annular zone. According to the four-axis asymmetric orthokeratology lens, four meridional lines of a lens body are determined based on a mean sagittal depth in the specific annular zone on the cornea. The four meridional lines divide the adaptation curve zone of the lens body into the four sector ring zones, and different curvatures are specifically set for each meridional line, to relatively independently design curvatures for curved surfaces of the four sector ring zones.

### CROSS-REFERENCE TO RELATED APPLICATIONS

This application claims priority to Chinese Patent Application No. 202410228718X with a filing date of Feb. 29, 2024 and Chinese Patent Application No. 202411253643.7 with a filing date of Sep. 6, 2024. The content of the aforementioned applications, including any intervening amendments thereto, is incorporated herein by reference.

### TECHNICAL FIELD

The present disclosure relates to the technical field of orthokeratology lenses, and in particular to a four-axis asymmetric orthokeratology lens and a manufacturing method.

### BACKGROUND

The orthokeratology lens is a rigid breathable lens for orthokeratology, and generally includes four curve zones, namely, a base curve zone, a reverse curve zone, an adaptation curve zone, and a peripheral curve zone. The adaptation curve zone is used to ensure that the orthokeratology lens is well positioned in a center of a cornea, to achieve ideal shaping effect. However, most adaptation curve zones of existing orthokeratology lenses have a spherical geometrical structure that is rotationally symmetric or a compound surface geometrical structure that is geometrically symmetric. Therefore, the existing orthokeratology lenses cannot be well adapted to most anterior surfaces of corneas that are asymmetric and irregular. As a result, the existing orthokeratology lenses are offset, leading to potential safety hazards.

The existing Chinese Patent Publication No. CN114740635A discloses a four-quadrant asymmetric orthokeratology lens. In this patent, a four-quadrant asymmetric design is used in the adaptation curve zone. However, in the four quadrants of the four-quadrant asymmetric orthokeratology lens, areas covering the corneas are the same, and the four meridional lines dividing the four quadrants are perpendicular to each other. As a result, the inner surfaces, transited in the four quadrants, of the four-quadrant asymmetric orthokeratology lens are not well fitted with the anterior surface of the cornea. In addition, the existing Chinese Patent Publication No. CN117192806A discloses a shaped four-quadrant asymmetric orthokeratology lens. In this patent, a shaped four-quadrant asymmetric design is used in the adaptation curve zone. However, division of the meridional lines is fixed, and not performed based on an actual corneal morphology. As a result, the inner surfaces, transited between the meridional lines, of the four-quadrant asymmetric orthokeratology lens are not well fitted with the anterior surface of the cornea. It can be learned that the existing orthokeratology lenses have problems that myopia prevention and control effect is poor due to offsetting of the orthokeratology lenses and cannot be effectively achieved.

Therefore, to resolve the above problem, the present disclosure provides a four-axis asymmetric orthokeratology lens that is more appropriate for a cornea of a wearer and a manufacturing method.

### SUMMARY OF PRESENT INVENTION

To resolve the above problems, the present disclosure provides a four-axis asymmetric orthokeratology lens and a manufacturing method.

To achieve the above objective of the present disclosure, the present disclosure provides a four-axis asymmetric orthokeratology lens. The four-axis asymmetric orthokeratology lens includes:
a base curve zone, and a reverse curve zone, an adaptation curve zone, and a peripheral curve zone that are successively formed outward from a periphery of the base curve zone; where
a plurality of meridional lines are defined on the four-axis asymmetric orthokeratology lens; a mean sagittal depth is calculated to define distances between measurement points on each of the meridional lines extended onto an inner surface of the adaptation curve zone and a horizontal surface of an apex of an inner surface of the four-axis asymmetric orthokeratology lens; among all meridional lines on the four-axis asymmetric orthokeratology lens, a meridional line with a smallest mean sagittal depth is defined as a flattest meridional line, a meridional line with a largest mean sagittal depth is defined as a steepest meridional line; among meridional lines having included angles with the flattest meridional line of not less than 90°, a meridional line having a smallest mean sagittal depth is defined as a secondary flat meridional line; among meridional lines having included angles with the steepest meridional line of not less than 90°, a meridional line having a largest mean sagittal depth is defined as a secondary steep meridional line; the adaptation curve zone is divided into four sector ring zones by the flattest meridional line, the secondary flat meridional line, the steepest meridional line, and the secondary steep meridional line;
a center of a circle of the base curve zone is configured to correspond to a center of a cornea, and a zone, corresponding to the adaptation curve zone, of the cornea is defined as a specific annular zone;
a curvature distribution of the flattest meridional line of the four-axis asymmetric orthokeratology lens on the inner surface of the adaptation curve zone is consistent with a curvature distribution of an outer surface of a flattest meridional line of the cornea in the specific annular zone;
a curvature distribution of the secondary flat meridional line of the four-axis asymmetric orthokeratology lens on the inner surface of the adaptation curve zone is consistent with a curvature distribution of an outer surface of a secondary flat meridional line of the cornea in the specific annular zone;
a curvature distribution of the steepest meridional line of the four-axis asymmetric orthokeratology lens on the inner surface of the adaptation curve zone is consistent with a curvature distribution of an outer surface of a steepest meridional line of the cornea in the specific annular zone; and
a curvature distribution of the secondary steep meridional line of the four-axis asymmetric orthokeratology lens on the inner surface of the adaptation curve zone is consistent with a curvature distribution of an outer surface of a secondary steep meridional line of the cornea in the specific annular zone.

Preferably, adjacent sector ring zones are connected in a gradual transition manner, and a curvature of each sector ring zone is gradually transited between edges of two meridional lines that divide the sector ring zone.

Preferably, when a difference between mean sagittal depths of adjacent meridional lines is greater than 45 μm, a zoning meridional line is defined between the adjacent meridional lines on the four-axis asymmetric orthokeratology lens, the zoning meridional line divides a sector ring zone formed between the adjacent meridional lines into two sector ring sub-zones, a curvature distribution of the zoning meridional line of the four-axis asymmetric orthokeratology lens on the inner surface of the adaptation curve zone is consistent with a curvature distribution of a zoning meridional line of the cornea on an anterior surface of the cornea in the specific annular zone.

Preferably, the zoning meridional line of the four-axis asymmetric orthokeratology lens is a meridional line having a mean sagittal depth of an intermediate value among all meridional lines in the sector ring zone where the zoning meridional line is located.

Preferably, the two sector ring sub-zones in the sector ring zone are connected in a gradual transition manner, and inner surfaces of the two sector ring sub-zones form a smooth curved surface.

Preferably, a curvature of the sector ring zone is gradually transited from edges of two meridional lines that divide the sector ring zone to an edge of the zoning meridional line in the sector ring zone.

The present disclosure further provides a method for manufacturing a four-axis asymmetric orthokeratology lens. The method includes:
S10, providing a to-be-adjusted lens body, where the to-be-adjusted lens body includes a base curve zone, and a reverse curve zone, an adaptation curve zone, and a peripheral curve zone that are successively formed outward from a periphery of the base curve zone;
S20, dividing the adaptation curve zone into four sector ring zones by a flattest meridional line, a secondary flat meridional line, a steepest meridional line, and a secondary steep meridional line, where the flattest meridional line, the secondary flat meridional line, the steepest meridional line, and the secondary steep meridional line pass through a center of a circle of the base curve zone, and the flattest meridional line, the secondary flat meridional line, the steepest meridional line, and the secondary steep meridional line are determined according to the following steps:

S21, obtaining a plurality of meridional lines of a cornea;
S22, enabling the center of a circle of the base curve zone of the to-be-adjusted lens body to correspond to a center of the cornea, and selecting a zone, corresponding to a specific annular zone, of the cornea as the adaptation curve zone of the to-be-adjusted lens body;
S23, for each meridional line on the cornea in the specific annular zone, calculating a mean sagittal depth between an anterior surface of the cornea and a horizontal surface of an apex of the cornea;
S24, obtaining a flattest meridional line of the cornea and a steepest meridional line of the cornea, where among all meridional lines of the cornea, the flattest meridional line of the cornea is a meridional line having a smallest mean sagittal depth, and the steepest meridional line of the cornea is a meridional line having a largest mean sagittal depth;
S25, obtaining a secondary flat meridional line of the cornea and a secondary steep meridional line of the cornea, where the secondary flat meridional line of the cornea is a meridional line having an included angle with the flattest meridional line of the cornea of not less than 90° and a smallest mean sagittal depth, and the secondary steep meridional line of the cornea is a meridional line having an included angle with the steepest meridional line of the cornea of not less than 90° and a largest mean sagittal depth;
S30, adjusting curvature distributions of the flattest meridional line of the to-be-adjusted lens body, the secondary flat meridional line of the to-be-adjusted lens body, the steepest meridional line of the to-be-adjusted lens body, the secondary steep meridional line of the to-be-adjusted lens body on an inner surface in the adaptation curve zone based on curvature distributions of the flattest meridional line of the cornea, the secondary flat meridional line of the cornea, the steepest meridional line of the cornea, and the secondary steep meridional line of the cornea on the anterior surface of the cornea in the specific annular zone, to obtain a four-axis asymmetric orthokeratology lens, where a curvature distribution of a flattest meridional line of the four-axis asymmetric orthokeratology lens on an inner surface of the adaptation curve zone is consistent with a curvature distribution of an outer surface of the flattest meridional line of the cornea in the specific annular zone; a curvature distribution of a secondary flat meridional line of the four-axis asymmetric orthokeratology lens on the inner surface of the adaptation curve zone is consistent with a curvature distribution of an outer surface of a secondary flat meridional line of the cornea in the specific annular zone; a curvature distribution of a steepest meridional line of the four-axis asymmetric orthokeratology lens on the inner surface of the adaptation curve zone is consistent with a curvature distribution of an outer surface of the steepest meridional line of the cornea in the specific annular zone; a curvature distribution of a secondary steep meridional line of the four-axis asymmetric orthokeratology lens on the inner surface of the adaptation curve zone is consistent with a curvature distribution of an outer surface of the secondary steep meridional line of the cornea in the specific annular zone; and adjacent sector ring zones are gradually transited.

Preferably, the method for manufacturing a four-axis asymmetric orthokeratology lens further includes:
S40, obtaining a zoning meridional line of the cornea, where when a difference between mean sagittal depths of adjacent meridional lines of the cornea is greater than 45 μm, the zoning meridional line of the cornea is a meridional line defined between adjacent meridional lines, and the zoning meridional line of the cornea divides a sector ring zone formed between the adjacent meridional lines into two sector ring sub-zones; and
S41, adjusting a zoning meridional line of the to-be-adjusted lens body based on a curvature distribution of the zoning meridional line of the cornea on the anterior surface of the cornea in the specific annular zone, to obtain the four-axis asymmetric orthokeratology lens, where a curvature distribution of a zoning meridional line of the four-axis asymmetric orthokeratology lens on the inner surface of the adaptation curve zone is consistent with a curvature distribution of the zoning meridional line of the cornea on an outer surface of the cornea in the specific annular zone; and adjacent sector ring sub-zones are gradually transited.

Preferably, the zoning meridional line of the four-axis asymmetric orthokeratology lens is a meridional line having a mean sagittal depth of an intermediate value among all meridional lines between the two adjacent meridional lines.

Preferably, the base curve zone, the reverse curve zone, the adaptation curve zone, and the peripheral curve zone are integrally molded from inside to outside.

Compared with the prior art, the present disclosure has the following beneficial effect:

1. According to the four-axis asymmetric orthokeratology lens, the four meridional lines of the lens body are determined based on the mean sagittal depth in the specific annular zone on the cornea. The flattest meridional line corresponds to a flattest zone of the cornea, the secondary flat meridional line corresponds to the secondary flat zone of the cornea, the steepest meridional line corresponds to a steepest zone of the cornea, and the secondary steep meridional line corresponds to a secondary steep zone of the cornea. The four meridional lines divide the adaptation curve zone of the lens body into the four sector ring zones, and different curvatures are specifically set for each meridional line, to relatively independently design curvatures for curved surfaces of the four sector ring zones. Therefore, the four-axis asymmetric orthokeratology lens is more appropriate for a wearer, and better adaptation is achieved. This is conducive to accurately performing personalized fitting for various asymmetric and irregular corneas.

2. The zoning meridional line is defined in a sector ring zone with a large span of the mean sagittal depth, to subdivide the sector ring zone with a large span of the mean sagittal depth into two sector ring sub-zones. Because the zoning meridional line is determined by taking a middle value of the mean sagittal depth of all meridional lines in a sector ring zone in which the zoning meridional line is located, a corneal topography of the wearer can be more accurately fitted. Therefore, each sector ring zone of the adaptation curve zone of the lens body is appropriately fitted with the cornea of the wearer. This further improves adaptation to the cornea, effectively prevents the lens body from offsetting, and optimizes shaping effect on the cornea.

The following describes the present disclosure with reference to accompanying drawings and embodiments.

### BRIEF DESCRIPTION OF THE DRAWINGS

FIG. 1 is a schematic diagram of an overall structure of a four-axis asymmetric orthokeratology lens at a visual angle according to the present disclosure;

FIG. 2 is a schematic diagram of a cross-sectional structure of a four-axis asymmetric orthokeratology lens at another visual angle according to the present disclosure;

FIG. 3 is a schematic diagram of a flattest meridional line, a steepest meridional line, a secondary flat meridional line, and a secondary steep meridional line of a four-axis asymmetric orthokeratology lens according to a present disclosure; and

FIG. 4 is a schematic diagram of calculation for a sagittal depth of one meridional line of a four-axis asymmetric orthokeratology lens according to the present disclosure.

Reference numerals: 1, base curve zone; 2, reverse curve zone; 3, adaptation curve zone; 31, first sector ring zone; 32, second sector ring zone; 33, third sector ring zone; 34, fourth sector ring zone; 35, zoning meridional line I; 36, zoning meridional line II; 37, zoning meridional line III; 38, zoning meridional line IV; 4, peripheral curve zone; 5, center of circle of base curve zone; 6, flattest meridional line; 7, steepest meridional line; 8, secondary flat meridional line; 9, secondary steep meridional line.

### DETAILED DESCRIPTION OF THE EMBODIMENTS

The following illustrates in details the present disclosure, such that those skilled in the art can realize the present disclosure. The following preferred embodiments are merely used as an example for description, other apparent variations are likewise conceivable to those skilled in the art. The basic principles of the present disclosure defined in the following description may be applied to other implementations, variations, improvements, equivalents, and other technical solutions that do not depart from the spirit and scope of the present disclosure.

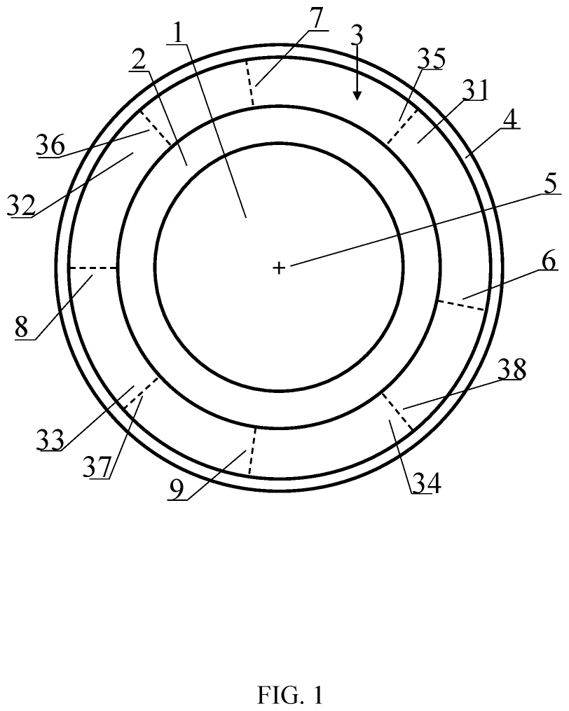

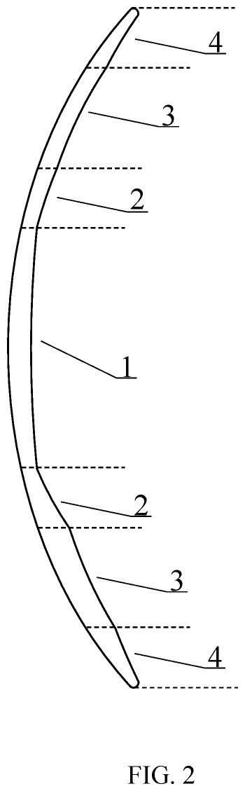

Referring to FIG. 1 and FIG. 2, the present disclosure provides a technical solution: A four-axis asymmetric orthokeratology lens includes

a circular arc sheet-shaped lens body. The lens body includes a base curve zone 1, a reverse curve zone 2, an adaptation curve zone 3, and a peripheral curve zone 4. The base curve zone 1, the reverse curve zone 2, the adaptation curve zone 3, and the peripheral curve zone 4 are integrally molded from inside to outside. The base curve zone 1 is an arc sheet-shaped structure located in a center zone of the lens body. The reverse curve zone 2 is a narrower annular structure surrounding the base curve zone 1. The adaptation curve zone 3 is a slightly wider annular structure surrounding the reverse curve zone 2. The peripheral curve zone 4 is a narrower annular structure surrounding the adaptation curve zone 3. As a central optical zone and a treatment zone, the base curve zone 1 is mainly used to flatten a surface of a corneal. Central tears are gathered in the reverse curve zone 2, to prompt the base curve zone 1 of a lens to flatten a center portion of the anterior surface of the cornea. The adaptation curve zone 3 is fitted with the cornea, to fix the lens and improve stability of the lens. The peripheral curve zone 4 is slightly warped at an edge, to smoothly exchange the tears.

The four-axis asymmetric orthokeratology lens includes a base curve zone 1, and a reverse curve zone 2, an adaptation curve zone 3, and a peripheral curve zone 4 that are successively formed outward from a periphery of the base curve zone 1.

During use, a center of a circle of the base curve zone 1 corresponds to a center of a cornea, and a zone, corresponding to the adaptation curve zone 3, of the cornea is defined as a specific annular zone.

Data relating to a plurality of meridional lines of the cornea is obtained, and the plurality of meridional lines of the cornea may divide the cornea into a plurality of equal sector zones. In the specific annular zone, a mean sagittal depth is present between an anterior surface of the cornea and a horizontal surface of an apex of the cornea; and among the plurality of meridional lines on the cornea, a meridional line having a smallest mean sagittal depth is a flattest meridional line of the cornea, a meridional line having largest mean sagittal depth is a steepest meridional line of the cornea, a meridional line having an included angle with the flattest meridional line of not less than 90° and smallest mean sagittal depth is a secondary flat meridional line of the cornea, and a meridional line having an included angle with the steepest meridional line of not less than 90° and largest mean sagittal depth is a secondary steep meridional line of the cornea. The specific annular zone is divided into four sector ring zones by the flattest meridional line of the cornea, the secondary flat meridional line of the cornea, the steepest meridional line of the cornea, and the secondary steep meridional line of the cornea.

Similarly, a plurality of meridional lines is defined on the four-axis asymmetric orthokeratology lens. A mean sagittal depth is calculated to define distances between measuring points on each of the meridional lines extended onto an inner surface of the adaptation curve zone 3 and a horizontal surface of an apex of an inner surface of the four-axis asymmetric orthokeratology lens. Among all meridional lines on the four-axis asymmetric orthokeratology lens, a meridional line with a smallest mean sagittal depth is a flattest meridional line 6 of the four-axis asymmetric orthokeratology lens, and a meridional line with a largest mean sagittal depth is a steepest meridional line 7 of the four-axis asymmetric orthokeratology lens. Among meridional lines having included angles with the flattest meridional line 6 of the four-axis asymmetric orthokeratology lens of not less than 90°, a meridional line having a smallest mean sagittal depth is a secondary flat meridional line 8 of the four-axis asymmetric orthokeratology lens, among meridional lines having included angles with the steepest meridional line 7 of the four-axis asymmetric orthokeratology lens of not less than 90°, a meridional line having a largest mean sagittal depth is a secondary steep meridional line 9 of the four-axis asymmetric orthokeratology lens. The adaptation curve zone 3 is divided into four sector ring zones by the flattest meridional line 6 of the four-axis asymmetric orthokeratology lens, the secondary flat meridional line 8 of the four-axis asymmetric orthokeratology lens, the steepest meridional line 7 of the four-axis asymmetric orthokeratology lens, and the secondary steep meridional line 9 of the four-axis asymmetric orthokeratology lens.

A curvature distribution of the flattest meridional line of the four-axis asymmetric orthokeratology lens on the inner surface of the adaptation curve zone is consistent with a curvature distribution of an outer surface of a flattest meridional line of the cornea in the specific annular zone.

A curvature distribution of a secondary flat meridional line of the four-axis asymmetric orthokeratology lens on the inner surface of the adaptation curve zone is consistent with a curvature distribution of an outer surface of a secondary flat meridional line of the cornea in the specific annular zone.

A curvature distribution of a steepest meridional line of the four-axis asymmetric orthokeratology lens on the inner surface of the adaptation curve zone is consistent with a curvature distribution of an outer surface of the steepest meridional line of the cornea in the specific annular zone;

A curvature distribution of the secondary steep meridional line of the four-axis asymmetric orthokeratology lens on the inner surface of the adaptation curve zone is consistent with a curvature distribution of an outer surface of a secondary steep meridional line of the cornea in the specific annular zone.

Preferably, on the four-axis asymmetric orthokeratology lens, adjacent sector ring zones are connected in a gradual transition manner, and inner surfaces of the adjacent sector ring zones form a smooth curved surface.

Specifically, referring to FIG. 1, on the four-axis asymmetric orthokeratology lens, the four sector ring zones are a first sector ring zone 31, a second sector ring zone 32, a third sector ring zone 33, and a fourth sector ring zone 34 respectively. The first sector ring zone 31 and the second sector ring zone 32 are zoned by the steepest meridional line 7 of the four-axis asymmetric orthokeratology lens, the first sector ring zone 31 and the second sector ring zone 32 are connected in a gradual transition manner, and inner surfaces of the first sector ring zone 31 and the second sector ring zone 32 form a smooth curved surface. The second sector ring zone 32 and the third sector ring zone 33 are zoned by the secondary flat meridional line 8 of the four-axis asymmetric orthokeratology lens, the second sector ring zone 32 is connected to the third sector ring zone 33 in a gradual transition manner, and inner surfaces of second sector ring zone 32 and the third sector ring zone 33 form a smooth curved surface. The third sector ring zone 33 and the fourth sector ring zone 34 are zoned by the secondary steep meridional line 9 of the four-axis asymmetric orthokeratology lens, the third sector ring zone 33 is connected to the fourth sector ring zone 34 in a gradual transition manner, and inner surfaces of the third sector ring zone 33 and the fourth sector ring zone 34 form a smooth curved surface. The fourth sector ring zone 34 and the first sector ring zone 31 are zoned by the flattest meridional line 6 of the four-axis asymmetric orthokeratology lens, the fourth sector ring zone 34 is connected to the first sector ring zone 31 in a gradual transition manner, and inner surfaces of the fourth sector ring zone 34 and the first sector ring zone 31 form a smooth curved surface.

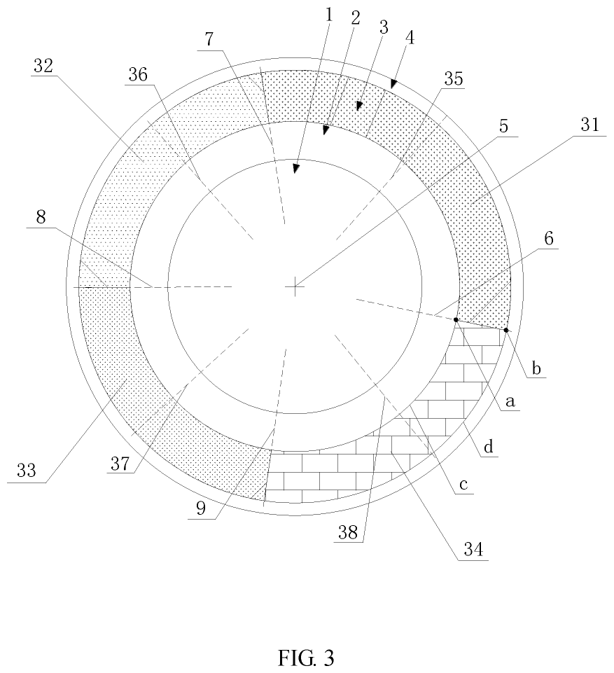

Referring to FIG. 3, two edges of the inner surface of the adaptation curve zone 3 in a radial direction are a curve zone edge c and a curve zone edge d respectively. The flattest meridional line 6 intersects with the curve zone edge c, to form an intersection point a, and the flattest meridional line 6 intersects with the curve zone edge d, to form an intersection point b. In the flattest meridional line 6 on an inner surface of the lens body, the intersection point a and the intersection point b as well as all points between the intersection point a and the intersection point b form a set. The set is referred to as a curve segment ab, and is the flattest meridional line 6 on the inner surface of the adaptation curve zone 3. A person skilled in the art may, by analogy, determine the secondary flat meridional line 8 of the inner surface of the adaptation curve zone 3, the steepest meridional line 7 of the inner surface of the adaptation curve zone 3, the secondary steep meridional line 9 of the inner surface of the adaptation curve zone 3, the flattest meridional line of the anterior surface in specific annular zone, the secondary flat meridional line of the anterior surface in the specific annular zone, the steepest meridional line of the anterior surface in the specific annular zone, and the secondary steep meridional line of the anterior surface in the specific annular zone.

The curve zone edges are edges at two sides of the inner surface of the adaptation curve zone 3, and the flattest meridional line 6 of the inner surface of the adaptation curve zone 3 is a line segment intercepted from the flattest meridional line 6 of the inner surface of the four-axis asymmetric orthokeratology lens via the two curve zone edges. In other words, the flattest meridional line 6 of the inner surface of the adaptation curve zone 3 has an end portion in a length direction defined on one of the curve zone edges.

It should be noted that the meridional line of the cornea is a straight line on the cornea. The meridional line of the cornea passes through the center of the cornea and has two line ends reaching an edge of the cornea. When the four-axis asymmetric orthokeratology lens is placed on the anterior surface of the cornea, the meridional line of the four-axis asymmetric orthokeratology lens is a straight line that is on the four-axis asymmetric orthokeratology lens and that corresponds to the meridional line of the cornea. The sagittal depth is a concept commonly used in geometry. For the four-axis asymmetric orthokeratology lens and the cornea, the sagittal depth can help to understand the design characteristics of the lens body, such as the thickness distribution of the lens body, the curvature of the lens body, and so on. The sagittal depth means a vertical distance from a reference plane to a point on a surface of a lens body or a cornea. For calculation of the mean sagittal depth on the cornea and on the lens body, each meridional line on the cornea has a plurality of measurement points on the anterior surface in the specific annular zone. Vertical distances from measurement points each to the horizontal surface of the apex of the anterior surface of the cornea are added, and then divided by the number of measurement points, to obtain the mean sagittal depth of the meridional lines of the cornea on the anterior surface in the specific annular zone. Each meridional line on the lens body has a plurality of measurement points on the inner surface of the adaptation curve zone 3. Vertical distances from measurement points each to the horizontal surface of the apex of the inner surface of the lens body are added, and then divided by the number of measurement points, to obtain the mean sagittal depth of the meridional lines of the lens body on the inner surface of the adaptation curve zone 3. On the four-axis asymmetric orthokeratology lens, a distance from the surface of the lens body to a specific reference plane may be obtained via an optical microscope or an interferometer.

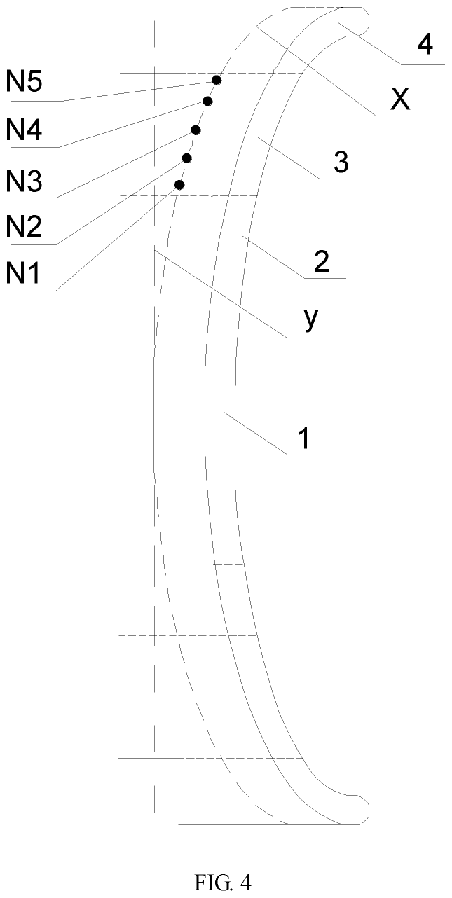

In an embodiment of the present disclosure, referring to FIG. 4, a meridional line x is defined on the lens body, and has a horizontal surface y of the apex. Five measurement points, namely, N1, N2, N3, N4, and N5 are defined on the meridional line x of the inner surface of the adaptation curve zone 3. Vertical distances from N1, N2, N3, N4, and N5 each to the horizontal surface y of the apex of the lens body are measured and added, and then divided by the number of 5 of measurement points, to obtain a mean sagittal depth of the meridional lines x of the lens body on the inner surface of the adaptation curve zone 3.

According to the four-axis asymmetric orthokeratology lens, the four meridional lines of the lens body are determined based on the mean sagittal depth in the specific annular zone on the cornea. The flattest meridional line 6 of the four-axis asymmetric orthokeratology lens corresponds to a flattest zone of the cornea, the secondary flat meridional line 8 of the four-axis asymmetric orthokeratology lens corresponds to the secondary flat zone of the cornea, the steepest meridional line 7 of the four-axis asymmetric orthokeratology lens corresponds to a steepest zone of the cornea, and the secondary steep meridional line 9 of the four-axis asymmetric orthokeratology lens corresponds to a secondary steep zone of the cornea. The four meridional lines of the four-axis asymmetric orthokeratology lens divide the adaptation curve zone 3 of the lens body into the four sector ring zones, and different curvatures are specifically set for each meridional line, to relatively independently design curvatures for curved surfaces of the four sector ring zones. Therefore, the four-axis asymmetric orthokeratology lens is more appropriate for a wearer, and better adaptation is achieved. This is conducive to accurately performing personalized fitting for various asymmetric and irregular corneas.

Preferably, the base curve zone 1, the reverse curve zone 2, the adaptation curve zone 3, and the peripheral curve zone 4 are integrally molded from inside to outside.

It should be noted that, in the present disclosure, the center of the cornea corresponds to the center of the circle 5 of the base curve zone. Therefore, the flattest meridional line 6 of the four-axis asymmetric orthokeratology lens, the secondary flat meridional line 8 of the four-axis asymmetric orthokeratology lens, the steepest meridional line 7 of the four-axis asymmetric orthokeratology lens, and the secondary steep meridional line 9 of the four-axis asymmetric orthokeratology lens penetrate through the center of the circle of the base curve zone 1.

Preferably, on the four-axis asymmetric orthokeratology lens, a curvature of each sector ring zone is gradually transited between edges of two meridional lines that divide the sector ring zone.

In an embodiment of the present disclosure, a curvature of the first sector ring zone 31 is directly and uniformly increased from an edge at which the flattest meridional line 6 of the four-axis asymmetric orthokeratology lens is located to an edge at which the steepest meridional line 7 of the four-axis asymmetric orthokeratology lens is located.

A curvature of the second sector ring zone 32 is directly and uniformly decreased from the edge at which the steepest meridional line 7 of the four-axis asymmetric orthokeratology lens is located to an edge at which the secondary flat meridional line 8 of the four-axis asymmetric orthokeratology lens is located.

A curvature of the third sector ring zone 33 is directly and uniformly increased from the edge at which the secondary flat meridional line 8 of the four-axis asymmetric orthokeratology lens is located to an edge at which the secondary steep meridional line 9 of the four-axis asymmetric orthokeratology lens is located.

A curvature of the fourth sector ring zone 34 is directly and uniformly decreased from the edge of the secondary steep meridional line 9 of the four-axis asymmetric orthokeratology lens is located to the edge at which the flattest meridional line 6 of the four-axis asymmetric orthokeratology lens is located.

To further improve adaptation to the cornea, effectively prevent the lens body from offsetting, and optimize shaping effect on the cornea, with regard to the four-axis asymmetric orthokeratology lens and the cornea, when a difference between mean sagittal depths of adjacent meridional lines is greater than 45 μm, a zoning meridional line of the four-axis asymmetric orthokeratology lens is defined between the adjacent meridional lines, the zoning meridional line of the four-axis asymmetric orthokeratology lens divides a sector ring zone formed between the adjacent meridional lines into two sector ring sub-zones,

Specifically, a difference between mean sagittal depths of the flattest meridional line 6 of the four-axis asymmetric orthokeratology lens and the steepest meridional line 7 of the four-axis asymmetric orthokeratology lens is greater than 45 m. A zoning meridional line I 35 of the four-axis asymmetric orthokeratology lens is defined between the flattest meridional line 6 of the four-axis asymmetric orthokeratology lens and the steepest meridional line 7 of the four-axis asymmetric orthokeratology lens. The first sector ring zone 31 is divided into two sector ring sub-zones via the zoning meridional line I 35 of the four-axis asymmetric orthokeratology lens, the two sector ring sub-zones are connected in a gradual transition manner, and vice versa, no zoning meridional line I 35 of the four-axis asymmetric orthokeratology lens is defined. When a difference between mean sagittal depths of the steepest meridional line 7 of the four-axis asymmetric orthokeratology lens and the secondary flat meridional line 8 of the four-axis asymmetric orthokeratology lens is greater than 45 μm, a zoning meridional line II 36 of the four-axis asymmetric orthokeratology lens is defined between the steepest meridional line 7 of the four-axis asymmetric orthokeratology lens and the secondary flat meridional line 8 of the four-axis asymmetric orthokeratology lens, the second sector ring zone 32 is divided into two sector ring sub-zones via the zoning meridional line II 36 of the four-axis asymmetric orthokeratology lens, the two sector ring sub-zones are connected in a gradual transition manner, and vice versa, no zoning meridional line II 36 of the four-axis asymmetric orthokeratology lens is defined. When a difference between mean sagittal depths of the secondary flat meridional line 8 of the four-axis asymmetric orthokeratology lens and the secondary steep meridional line 9 of the four-axis asymmetric orthokeratology lens is greater than 45 μm, a zoning meridional line III 37 of the four-axis asymmetric orthokeratology lens is defined between the secondary flat meridional line 8 of the four-axis asymmetric orthokeratology lens and the secondary steep meridional line 9 of the four-axis asymmetric orthokeratology lens, the third sector ring zone 33 is divided into two sector ring sub-zones via the zoning meridional line III 37 of the four-axis asymmetric orthokeratology lens, the two sector ring sub-zones are connected in a gradual transition manner, and vice versa, no zoning meridional line 11137 of the four-axis asymmetric orthokeratology lens is defined. When a difference between mean sagittal depths of the secondary steep meridional line 9 of the four-axis asymmetric orthokeratology lens and the flattest meridional line 6 of the four-axis asymmetric orthokeratology lens is greater than 45 μm, a zoning meridional line IV 38 of the four-axis asymmetric orthokeratology lens is defined between the secondary steep meridional line 9 of the four-axis asymmetric orthokeratology lens and the flattest meridional line 6 of the four-axis asymmetric orthokeratology lens, the fourth sector ring zone 34 is divided into two sector ring sub-zones via the zoning meridional line IV 38 of the four-axis asymmetric orthokeratology lens, the two sector ring sub-zones are connected in a gradual transition manner, and vice versa, no zoning meridional line IV 38 of the four-axis asymmetric orthokeratology lens is defined.

Preferably, the zoning meridional line of the four-axis asymmetric orthokeratology lens is a meridional line having a mean sagittal depth of an intermediate value among all meridional lines where the zoning meridional line is located.

In an embodiment of the present disclosure, the zoning meridional line of the four-axis asymmetric orthokeratology lens may include a zoning meridional line I 35, a zoning meridional line II 36, a zoning meridional line III 37, and a zoning meridional line IV 38.

The zoning meridional line I 35 is a meridional line that is selected from all meridional lines between the flattest meridional line 6 of the four-axis asymmetric orthokeratology lens and the steepest meridional line 7 of the four-axis asymmetric orthokeratology lens and that has an intermediate value of mean sagittal depth among all the selected meridional lines.

The zoning meridional line II 36 is a meridional line that is selected from all meridional lines between the steepest meridional line 7 of the four-axis asymmetric orthokeratology lens and the secondary flat meridional line 8 of the four-axis asymmetric orthokeratology lens and that has as an intermediate value of mean sagittal depth among all the selected meridional lines.

The zoning meridional line III 37 is a meridional line that is selected from all meridional lines between the secondary flat meridional line 8 of the four-axis asymmetric orthokeratology lens and the secondary steep meridional line 9 of the four-axis asymmetric orthokeratology lens and that has an intermediate value of mean sagittal depth among all the selected meridional lines.

The zoning meridional line IV 38 is a meridional line that is selected from all meridional lines between the secondary steep meridional line 9 of the four-axis asymmetric orthokeratology lens and the flattest meridional line 6 of the four-axis asymmetric orthokeratology lens and that has an intermediate value of mean sagittal depth among all the selected meridional lines.

Preferably, the two sector ring sub-zones in the sector ring zone of the four-axis asymmetric orthokeratology lens are connected in a gradual transition manner, and inner surfaces of the two sector ring sub-zones form a smooth curved surface. In other words, a curvature value of the inner surface of the sector ring sub-zone uniformly increases or decreases along a circumferential direction of the adaptation curve zone 3.

Preferably, a curvature of the sector ring zone of the four-axis asymmetric orthokeratology lens is gradually transited from edges of two meridional lines that divide the sector ring zone to an edge of the zoning meridional line in the sector ring zone.

In one embodiment of the present disclosure, a curvature value of the inner surface of the first sector ring zone 31 is uniformly increased first from the edge at which the flattest meridional line 6 of the four-axis asymmetric orthokeratology lens is located to an edge at which the zoning meridional line I 35 is located, and then uniformly increased from the edge at which the zoning meridional line I 35 is located to the edge at which the steepest meridional line 7 of the four-axis asymmetric orthokeratology lens is located.

A curvature value of the inner surface of the second sector ring zone 32 is uniformly decreased first from the edge at which the steepest meridional line 7 of the four-axis asymmetric orthokeratology lens is located to an edge at which the zoning meridional line II 36 is located, and then uniformly decreased from the edge at which the zoning meridional line II 36 is located to the edge at which the secondary flat meridional line 8 of the four-axis asymmetric orthokeratology lens is located.

A curvature value of the inner surface of the third sector ring zone 33 is uniformly increased from the edge at which the secondary flat meridional line 8 of the four-axis asymmetric orthokeratology lens is located to an edge at which the zoning meridional line III 37 is located, and then uniformly increased from the edge at which the zoning meridional line III 37 is located to the edge at which the secondary steep meridional line 9 of the four-axis asymmetric orthokeratology lens is located.

A curvature value of the inner surface of the fourth sector ring zone 34 is uniformly decreased first from the edge at which the secondary steep meridional line 9 of the four-axis asymmetric orthokeratology lens is located to an edge at which the zoning meridional line IV 38 is located, and then uniformly decreased from the edge at which the zoning meridional line IV 38 is located to the edge at which the flattest meridional line 6 of the four-axis asymmetric orthokeratology lens is located.

Specifically, a curvature value of a portion, closer to the flattest meridional line 6 of the four-axis asymmetric orthokeratology lens, of the inner surface of the first sector ring zone 31 is smaller, that is, flatter, and a curvature value of a portion, closer to the steepest meridional line 7 of the four-axis asymmetric orthokeratology lens, of the inner surface of the first sector ring zone 31 is larger, that is, steeper. A curvature value of the inner surface of the second sector ring zone 32 is uniformly decreased first from the edge at which the steepest meridional line 7 of the four-axis asymmetric orthokeratology lens is located to an edge at which the zoning meridional line II 36 is located, and then uniformly decreased from the edge at which the zoning meridional line II 36 is located to the edge at which the secondary flat meridional line 8 of the four-axis asymmetric orthokeratology lens is located. A curvature value of a portion, closer to the steepest meridional line 7 of the four-axis asymmetric orthokeratology lens, of the inner surface of the second sector ring zone 32 is larger, that is, steeper, and a curvature value of a portion, closer to the secondary flat meridional line 8 of the four-axis asymmetric orthokeratology lens, of the inner surface of the second sector ring zone 32 is smaller, that is, flatter. A curvature value of the inner surface of the third sector ring zone 33 is uniformly increased from the edge at which the secondary flat meridional line 8 of the four-axis asymmetric orthokeratology lens is located to an edge at which the zoning meridional line III 37 is located, and then uniformly increased from the edge at which the zoning meridional line III 37 is located to the edge at which the secondary steep meridional line 9 of the four-axis asymmetric orthokeratology lens is located. A curvature value of a portion, closer to the secondary flat meridional line 8 of the four-axis asymmetric orthokeratology lens, of the inner surface of the third sector ring zone 33 is smaller, that is, flatter, and a curvature value of a portion, closer to the secondary steep meridional line 9 of the four-axis asymmetric orthokeratology lens, of the inner surface of the third sector ring zone 33 is larger, that is, steeper. A curvature value of the inner surface of the fourth sector ring zone 34 is uniformly decreased first from the edge at which the secondary steep meridional line 9 of the four-axis asymmetric orthokeratology lens is located to an edge at which the zoning meridional line IV 38 is located, and then uniformly decreased from the edge at which the zoning meridional line IV 38 is located to the edge at which the flattest meridional line 6 of the four-axis asymmetric orthokeratology lens is located, A curvature value of a portion, closer to the secondary steep meridional line 9 of the four-axis asymmetric orthokeratology lens, of the inner surface of the fourth sector ring zone 34 is larger, that is, steeper, and a curvature value of a portion, closer to the flattest meridional line 6 of the four-axis asymmetric orthokeratology lens, of the inner surface of the fourth sector ring zone 34 is smaller, that is, flatter.

Preferably, curvature distributions of the zoning meridional line I 35 of the four-axis asymmetric orthokeratology lens, the zoning meridional line II 36 of the four-axis asymmetric orthokeratology lens, the zoning meridional line III 37 of the four-axis asymmetric orthokeratology lens, and the zoning meridional line IV 38 of the four-axis asymmetric orthokeratology lens on the inner surface of the adaptation curve zone 3 are consistent with the curvature distribution of the zoning meridional line of the cornea on the anterior surface in the specific annular zone.

For the four-axis asymmetric orthokeratology lens, the zoning meridional line is defined in a sector ring zone with a large span of the mean sagittal depth, to subdivide the sector ring zone with a large span of the mean sagittal depth into two sector ring sub-zones. Because the zoning meridional line is determined by taking a middle value of the mean sagittal depth of all meridional lines in a sector ring zone in which the zoning meridional line is located, a corneal topography of the wearer can be more accurately fitted. Therefore, each sector ring zone of the adaptation curve zone 3 of the lens body is appropriately fitted with the cornea of the wearer. This further improves adaptation to the cornea, effectively prevents the lens body from offsetting, and optimizes shaping effect on the cornea. The sector ring zone having a large span of the mean sagittal depth means that a difference between mean sagittal depths of two adjacent meridional lines is greater than 45 km.

Preferably, the method for using the four-axis asymmetric orthokeratology lens further includes:

During wearing, the center of the cornea is enabled to correspond to the center of the circle of the base curve zone 5, the four-axis asymmetric orthokeratology lens is directly placed on the cornea, and the anterior surface of the cornea is in contact with the inner surface of the four-axis asymmetric orthokeratology lens. Because tears exist between the cornea and the four-axis asymmetric orthokeratology lens, and degrees of curvatures of the meridional lines of the adaptation curve zone 3 of the four-axis asymmetric orthokeratology lens are different, the four-axis asymmetric orthokeratology lens slides on the cornea to a stable state. In this stable state, the anterior surface of the cornea is fitted to the inner surface of the four-axis asymmetric orthokeratology lens, the meridional line of the cornea is aligned to the meridional line of the four-axis asymmetric orthokeratology lens, and then the four-axis asymmetric orthokeratology lens applies a pressure on the cornea, to achieve shaping of the cornea

The present disclosure further provides a manufacturing method. The four-axis asymmetric orthokeratology lens is manufactured using the manufacturing method. The manufacturing method includes the following steps.

S10: Ato-be-adjusted lens body is provided. Tthe to-be-adjusted lens body includes a base curve zone 1, and a reverse curve zone 2, an adaptation curve zone 3, and a peripheral curve zone 4 that are successively formed outward from a periphery of the base curve zone 1.

S20: The adaptation curve zone 3 is divided into four sector ring zones by a flattest meridional line 6, a secondary flat meridional line 8, a steepest meridional line 7, and a secondary steep meridional line 9. The flattest meridional line 6, the secondary flat meridional line 8, the steepest meridional line 7, and the secondary steep meridional line 9 pass through a center of a circle of the base curve zone 1. The flattest meridional line 6, the secondary flat meridional line 8, the steepest meridional line 7, and the secondary steep meridional line 9 are determined according to the following steps.

S21: A plurality of meridional lines of a cornea are obtained.

S22: The center of a circle of the base curve zone 1 of the to-be-adjusted lens body is enabled to correspond to a center of the cornea, and a zone, corresponding to a specific annular zone, of the cornea is selected as the adaptation curve zone 3 of the to-be-adjusted lens body.

S23: For each meridional line on the cornea in the specific annular zone, a mean sagittal depth between an anterior surface of the cornea and a horizontal surface of an apex of the cornea is calculated.

S24: A flattest meridional line of the cornea and a steepest meridional line of the cornea are obtained. Among all meridional lines of the cornea, the flattest meridional line of the cornea is a meridional line having a smallest mean sagittal depth, and the steepest meridional line of the cornea is a meridional line having a largest mean sagittal depth.

S25: A secondary flat meridional line of the cornea and a secondary steep meridional line of the cornea are obtained. The secondary flat meridional line of the cornea is a meridional line having an included angle with the flattest meridional line of the cornea of not less than 90° and a smallest mean sagittal depth, and the secondary steep meridional line of the cornea is a meridional line having an included angle with the steepest meridional line of the cornea of not less than 90° and a largest mean sagittal depth.

S30: Curvature distributions of the flattest meridional line 6 of the to-be-adjusted lens body, the secondary flat meridional line 8 of the to-be-adjusted lens body, the steepest meridional line 7 of the to-be-adjusted lens body, the secondary steep meridional line 9 of the to-be-adjusted lens body on an inner surface in the adaptation curve zone 3 are adjusted based on curvature distributions of the flattest meridional line of the cornea, the secondary flat meridional line of the cornea, the steepest meridional line of the cornea, and the secondary steep meridional line of the cornea on the anterior surface of the cornea in the specific annular zone, to obtain a four-axis asymmetric orthokeratology lens. A curvature distribution of a flattest meridional line of the four-axis asymmetric orthokeratology lens on an inner surface of the adaptation curve zone is consistent with a curvature distribution of an outer surface of the flattest meridional line of the cornea in the specific annular zone. A curvature distribution of a secondary flat meridional line of the four-axis asymmetric orthokeratology lens on the inner surface of the adaptation curve zone is consistent with a curvature distribution of an outer surface of a secondary flat meridional line of the cornea in the specific annular zone. A curvature distribution of a steepest meridional line of the four-axis asymmetric orthokeratology lens on the inner surface of the adaptation curve zone is consistent with a curvature distribution of an outer surface of the steepest meridional line of the cornea in the specific annular zone. A curvature distribution of a secondary steep meridional line of the four-axis asymmetric orthokeratology lens on the inner surface of the adaptation curve zone is consistent with a curvature distribution of an outer surface of the secondary steep meridional line of the cornea in the specific annular zone. Adjacent sector ring zones are gradually transited.

The manufacturing method further includes:

S40: A zoning meridional line of the cornea is obtained. When a difference between mean sagittal depths of adjacent meridional lines of the cornea is greater than m, the zoning meridional line of the cornea is a meridional line defined between adjacent meridional lines, and the zoning meridional line of the cornea divides a sector ring zone formed between the adjacent meridional lines into two sector ring sub-zones

S41: A zoning meridional line of the to-be-adjusted lens body is adjusted based on a curvature distribution of the zoning meridional line of the cornea on the anterior surface of the cornea in the specific annular zone, to obtain the four-axis asymmetric orthokeratology lens. A curvature distribution of a zoning meridional line of the four-axis asymmetric orthokeratology lens on the inner surface of the adaptation curve zone 3 is consistent with a curvature distribution of the zoning meridional line of the cornea on an outer surface of the cornea in the specific annular zone; and adjacent sector ring sub-zones are gradually transited.

Preferably, the zoning meridional line of the four-axis asymmetric orthokeratology lens is a meridional line having a mean sagittal depth of an intermediate value among all meridional lines between the two adjacent meridional lines.

In conclusion, according to the four-axis asymmetric orthokeratology lens provided in the present disclosure, the four meridional lines of the lens body are determined based on the mean sagittal depth in the specific annular zone on the cornea. The four meridional lines divide the adaptation curve zone 3 of the lens body into the four sector ring zones, and different curvatures are specifically set for each meridional line, to relatively independently design curvatures for curved surfaces of the four sector ring zones. Further, the zoning meridional line is defined in a sector ring zone having a large span of the mean sagittal depth, to subdivide the sector ring zone having a large span of the mean sagittal depth into two sector ring sub-zones. Therefore, a corneal topography of the wearer can be more accurately fitted, and the four-axis asymmetric orthokeratology lens is more appropriate for the cornea of the wearer, so as to achieve better adaptation.

The above embodiments are only used to illustrate the technical ideas and features of the present disclosure, such that those skilled in the art can understand the content of the present disclosure and implement the present disclosure accordingly. The scope of the present disclosure is not limited by the above embodiments, that is, any equivalent changes or modifications made to the spirit disclosed by the present disclosure still fall within the scope of the present disclosure.

What is claimed is:

1. A four-axis asymmetric orthokeratology lens, comprising:
a base curve zone (1), and a reverse curve zone (2), an adaptation curve zone (3), and a peripheral curve zone (4) that are successively formed outward from a periphery of the base curve zone (1), wherein
a plurality of meridional lines are defined on the four-axis asymmetric orthokeratology lens; a mean sagittal depth is calculated to define distances between measurement points on each of the meridional lines extended onto an inner surface of the adaptation curve zone (3) and a horizontal surface of an apex of an inner surface of the four-axis asymmetric orthokeratology lens; among the plurality of meridional lines on the four-axis asymmetric orthokeratology lens, a meridional line with a smallest mean sagittal depth is defined as a flattest meridional line (6), a meridional line with a largest mean sagittal depth is a defined as steepest meridional line (7); among meridional lines having included angles with the flattest meridional line (6) of not less than 90°, a meridional line having a smallest mean sagittal depth is defined as a secondary flat meridional line (8); among meridional lines having included angles with the steepest meridional line (7) of not less than 90°, a meridional line having a largest mean sagittal depth is defined as a secondary steep meridional line (9); the adaptation curve zone (3) is divided into four sector ring zones by the flattest meridional line (6), the secondary flat meridional line (8), the steepest meridional line (7), and the secondary steep meridional line (9);
a center of a circle of the base curve zone (1) is configured to correspond to a center of a cornea, and a zone, corresponding to the adaptation curve zone (3), of the cornea is defined as a specific annular zone;
a curvature distribution of the flattest meridional line of the four-axis asymmetric orthokeratology lens on the inner surface of the adaptation curve zone is consistent with a curvature distribution of an outer surface of a flattest meridional line of the cornea in the specific annular zone;
a curvature distribution of the secondary flat meridional line of the four-axis asymmetric orthokeratology lens on the inner surface of the adaptation curve zone is consistent with a curvature distribution of an outer surface of a secondary flat meridional line of the cornea in the specific annular zone;
a curvature distribution of the steepest meridional line of the four-axis asymmetric orthokeratology lens on the inner surface of the adaptation curve zone is consistent with a curvature distribution of an outer surface of a steepest meridional line of the cornea in the specific annular zone; and
a curvature distribution of the secondary steep meridional line of the four-axis asymmetric orthokeratology lens on the inner surface of the adaptation curve zone is consistent with a curvature distribution of an outer surface of a secondary steep meridional line of the cornea in the specific annular zone.

2. The four-axis asymmetric orthokeratology lens according to claim 1, wherein adjacent two of the four sector ring zones are connected in a gradual transition manner, and a curvature of each of the four sector ring zones is gradually transited between edges of two meridional lines that divide the sector ring zone.

3. The four-axis asymmetric orthokeratology lens according to claim 1, wherein when a difference between mean sagittal depths of adjacent meridional lines is greater than 45 μm, a zoning meridional line is defined between the adjacent meridional lines on the four-axis asymmetric orthokeratology lens, the zoning meridional line divides a sector ring zone formed between the adjacent meridional lines into two sector ring sub-zones, a curvature distribution of the zoning meridional line of the four-axis asymmetric orthokeratology lens on the inner surface of the adaptation curve zone (3) is consistent with a curvature distribution of a zoning meridional line of the cornea on an anterior surface of the cornea in the specific annular zone.

4. The four-axis asymmetric orthokeratology lens according to claim 3, wherein the zoning meridional line of the four-axis asymmetric orthokeratology lens is a meridional line having a mean sagittal depth of an intermediate value among all meridional lines in the sector ring zone where the zoning meridional line is located.

5. The four-axis asymmetric orthokeratology lens according to claim 3, wherein the two sector ring sub-zones in the sector ring zone are connected in a gradual transition manner, and inner surfaces of the two sector ring sub-zones form a smooth curved surface.

6. The four-axis asymmetric orthokeratology lens according to claim 3, wherein a curvature of the sector ring zone is gradually transited from edges of two meridional lines that divide the sector ring zone to an edge of the zoning meridional line in the sector ring zone.

7. A method for manufacturing a four-axis asymmetric orthokeratology lens, comprising:
S10, providing a to-be-adjusted lens body, wherein the to-be-adjusted lens body comprises a base curve zone (1), and a reverse curve zone (2), an adaptation curve zone (3), and a peripheral curve zone (4) that are successively formed outward from a periphery of the base curve zone (1);
S20, dividing the adaptation curve zone (3) into four sector ring zones by a flattest meridional line (6), a secondary flat meridional line (8), a steepest meridional line (7), and a secondary steep meridional line (9), wherein the flattest meridional line (6), the secondary flat meridional line (8), the steepest meridional line (7), and the secondary steep meridional line (9) pass through a center of a circle of the base curve zone (1), and the flattest meridional line (6), the secondary flat meridional line (8), the steepest meridional line (7), and the secondary steep meridional line (9) are determined according to the following steps:
S21, obtaining a plurality of meridional lines of a cornea;
S22, enabling the center of a circle of the base curve zone (1) of the to-be-adjusted lens body to correspond to a center of the cornea, and selecting a zone, corresponding to a specific annular zone, of the cornea as the adaptation curve zone (3) of the to-be-adjusted lens body;
S23, for each meridional line on the cornea in the specific annular zone, calculating a mean sagittal depth between an anterior surface of the cornea and a horizontal surface of an apex of the cornea;
S24, obtaining a flattest meridional line of the cornea and a steepest meridional line of the cornea, wherein among the plurality of meridional lines of the cornea, the flattest meridional line of the cornea is a meridional line having a smallest mean sagittal depth, and the steepest meridional line of the cornea is a meridional line having a largest mean sagittal depth;
S25, obtaining a secondary flat meridional line of the cornea and a secondary steep meridional line of the cornea, wherein the secondary flat meridional line of the cornea is a meridional line having an included angle with the flattest meridional line of the cornea of not less than 90° and a smallest mean sagittal depth, and the secondary steep meridional line of the cornea is a meridional line having an included angle with the steepest meridional line of the cornea of not less than 90° and a largest mean sagittal depth;
S30, adjusting curvature distributions of the flattest meridional line (6) of the to-be-adjusted lens body, the secondary flat meridional line (8) of the to-be-adjusted lens body, the steepest meridional line (7) of the to-be-adjusted lens body, the secondary steep meridional line (9) of the to-be-adjusted lens body on an inner surface in the adaptation curve zone (3) based on curvature distributions of the flattest meridional line of the cornea, the secondary flat meridional line of the cornea, the steepest meridional line of the cornea, and the secondary steep meridional line of the cornea on the anterior surface of the cornea in the specific annular zone, to obtain a four-axis asymmetric orthokeratology lens, wherein a curvature distribution of a flattest meridional line of the four-axis asymmetric orthokeratology lens on an inner surface of the adaptation curve zone is consistent with a curvature distribution of an outer surface of the flattest meridional line of the cornea in the specific annular zone; a curvature distribution of a secondary flat meridional line of the four-axis asymmetric orthokeratology lens on the inner surface of the adaptation curve zone is consistent with a curvature distribution of an outer surface of a secondary flat meridional line of the cornea in the specific annular zone; a curvature distribution of a steepest meridional line of the four-axis asymmetric orthokeratology lens on the inner surface of the adaptation curve zone is consistent with a curvature distribution of an outer surface of the steepest meridional line of the cornea in the specific annular zone; a curvature distribution of a secondary steep meridional line of the four-axis asymmetric orthokeratology lens on the inner surface of the adaptation curve zone is consistent with a curvature distribution of an outer surface of the secondary steep meridional line of the cornea in the specific annular zone; and adjacent sector ring zones are gradually transited.

8. The method according to claim 7, further comprising:
S40, obtaining a zoning meridional line of the cornea, wherein when a difference between mean sagittal depths of adjacent meridional lines of the cornea is greater than 45 μm, the zoning meridional line of the cornea is a meridional line defined between the adjacent meridional lines, and the zoning meridional line of the cornea divides a sector ring zone formed between the adjacent meridional lines into two sector ring sub-zones; and
S41, adjusting a zoning meridional line of the to-be-adjusted lens body based on a curvature distribution of the zoning meridional line of the cornea on the anterior surface of the cornea in the specific annular zone, to obtain the four-axis asymmetric orthokeratology lens, wherein a curvature distribution of a zoning meridional line of the four-axis asymmetric orthokeratology lens on the inner surface of the adaptation curve zone (3) is consistent with a curvature distribution of the zoning meridional line of the cornea on an outer surface of the cornea in the specific annular zone; and adjacent sector ring sub-zones are gradually transited.

9. The method according to claim 8, wherein the zoning meridional line of the four-axis asymmetric orthokeratology lens is a meridional line having a mean sagittal depth of an intermediate value among all meridional lines between the two adjacent meridional lines.

10. The method according to claim 7, wherein the base curve zone (1), the reverse curve zone (2), the adaptation curve zone (3), and the peripheral curve zone (4) are integrally molded from inside to outside.
